(19) United States
(12) Patent Application Publication
(10) Pub. No.: US 2024/0277947 A1
(43) Pub. Date: Aug. 22, 2024

(54) ADAPTER FOR VAPORIZATION ATTACHMENTS

(71) Applicant: Kind Distribution LLC, Austin, TX (US)

(72) Inventors: Patrick Bissen, Austin, TX (US); Elie Atalla, Harrington Park, NJ (US)

(73) Assignee: Kind Distribution LLC, Austin, TX (US)

(21) Appl. No.: 18/385,883

(22) Filed: Oct. 31, 2023

Related U.S. Application Data
(60) Provisional application No. 63/485,668, filed on Feb. 17, 2023.

(51) Int. Cl. A61M 11/04; A61M 16/00

(52) U.S. Cl. CPC A61M 11/042; A61M 16/0003; A61M 2016/0018; A61M 2205/36

(57) ABSTRACT

The invention disclosed here is an Adapter for Vaporizer Attachments. The device connects any attachment with a tubular end to a vaporizer which has a segregated airpath and power button on one end and the heating chamber on the opposite end. The invention provides a substantially airtight connection between the air channel formed within the tubular end of the attachment and the airpath in the Vaporizer. Furthermore, the invention provides a connection between the attachment and the vaporizer's power button, whereby a user of the invention can depress the power button simply by pushing the attachment down towards the center of the vaporizer.

### CLAIM OF PRIORITY

This application claims priority to U.S. Provisional Utility Application No. 63/485,668 filed Feb. 17, 2023.

### FIELD OF INVENTION

This invention relates to the classification of Devices for Introducing Media Into, or Onto, the Body and one or more sub-classifications for Inhalators or Sprayers or Atomisers Specially Adapted for Therapeutic Purposes. Specifically, this invention is an adapter allowing an attachment to be mated with a vaporizer.

### BACKGROUND OF INVENTION

Electronic vaporization devices are commonly used to vaporize herbs, tobacco or other botanical products for the purpose of inhaling the released vapors. Otherwise referred to as personal vaporizers (“Vaporizers”) such devices typically have at least the following components: a power source, a power button, a heating element, a heating chamber, a mouthpiece and an airpath that connects the heating chamber to the mouthpiece.

Vaporizers are typically designed to work with one or more standard mouthpieces. However, many users prefer to use alternative Attachments, such as a glass tube, a water pipe adapter, a bent or curved tube or a mini bubbler (which holds water and forces the inhaled vapors to pass through the water before being inhaled). Regardless of the material or features that are desired and offered by such Attachments, they all have a common connection point: a tubular end. Attaching the tubular end to the rest of the Vaporizer is a problem in need of a solution.

In addition, when the tubular end of the Attachment is connected to the Vaporizer it blocks access to the power button in many Vaporizers currently on the market. This is a problem because it makes it more difficult to operate the Vaporizer after an attachment has been connected. Therefore, there exists a need for a device that can simultaneously connect a tubular Attachment to the Vaporizer's body, create a continuous seal between the Attachment and the Vaporizer's Airpath and facilitate the continuous operation of the Vaporizer's power button.

No such solution exists today.

### SUMMARY OF THE INVENTION

This summary is intended to disclose the present invention, an Adapter for Vaporizer Attachments. The embodiments and descriptions are used to illustrate the invention and its utility, and are not intended to limit the invention or its use. In the illustrated embodiment, the Adapter for Vaporizer Attachments is shown in conjunction with attachments for vaporizers.

A Vaporizer typically comes with a single style of mouthpiece. For users wishing to use their Vaporizer with a glass tube, a water pipe adapter, a bent or curved tube or a mini bubbler (which holds water and forces the inhaled vapors to pass through the water before being inhaled), a novel Adapter is disclosed in this Application. All of the Attachments have a tubular end. The tubular end fits in an opening in Adapter called the Attachment Interface. The Attachment Interface is designed to form an air-tight seal with the tubular end of an Attachment. It does this by having an elastomeric inner surface that is specifically sized so as to make a nominal interference fit with the tube on the Attachment.

The Adapter fits into the base of the Vaporizer, replacing the original mouthpiece. The Adapter creates a continuous air path between the Attachment and the airpath of the Vaporizer leading to the heating chamber. The Adapter creates an air-tight seal with the base of the Vaporizer. The Adapter has a rigid insert that terminates in an actuator. By pressing on the Attachment, a user is able to cycle a power button by transmitting force through the rigid insert and actuator to the power button. This allows the user to get the full benefits of the Vaporizer while using an Attachment rather than the manufacturer's mouthpiece.

### BRIEF DESCRIPTION OF THE DRAWINGS

The present invention is illustrated with 7 figures on 6 sheets.

FIG. 1 is a perspective view of a prior art vaporizer. FIG. 2 is a cross-section side view of a prior art vaporizer, showing the internal structure. FIG. 3 is a perspective view of a prior art vaporizer showing a removable feature.

FIG. 4 is a top perspective view of a prior art vaporizer base used in conjunction with the Adapter for Vaporizer Attachments. FIG. 5 is a bottom perspective view of a prior art vaporizer base used in conjunction with the Adapter for Vaporization Attachments.

FIG. 6A is a bottom perspective view of a rigid insert. FIG. 6B is a side view of a rigid insert. FIG. 6C is a top perspective view of a rigid insert.

FIG. 7 is a cross-section side view of the vaporizer interfaced with the Adapter for Vaporization Attachments.

### DETAILED DESCRIPTION OF THE DRAWINGS

The following descriptions are not meant to limit the invention, but rather to add to the summary of invention, and illustrate the present invention, by offering and illustrating various embodiments of the present invention, an Adapter for Vaporization Attachments 10. While embodiments of the invention are illustrated and described, the embodiments herein do not represent all possible forms of the invention. Rather, the descriptions, illustrations, and embodiments are intended to teach and inform one skilled in the art without limiting the scope of the invention.

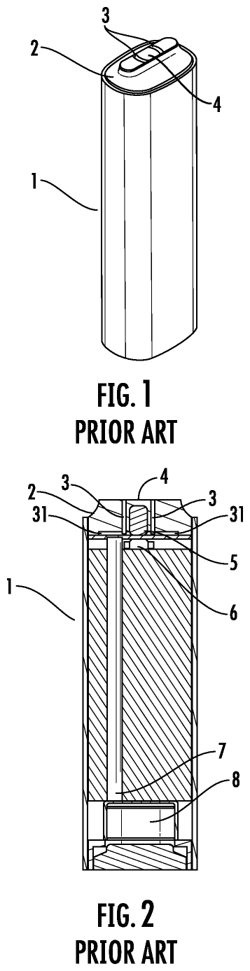

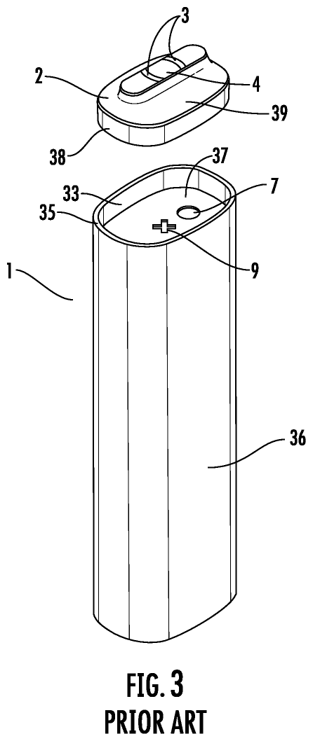

The Adapter for Vaporizer Attachments 1 connects any attachment with a tubular end 11 (“Attachment”) to a vaporizer 1 with an interface similar to the example shown in FIGS. 1 through 3, heretofore referred to as Vaporizers 1. In addition, the invention provides a substantially airtight connection between the air channel formed within the tubular end of the Attachment 11 and the Airpath 7 in the Vaporizer 1. Furthermore, the invention provides a connection between the Attachment 12 and the Vaporizer's power button 6, whereby a user of the invention 10 can depress the power button 6 simply by pushing the Attachment 11 down towards the center of the Vaporizer 1.

The claimed invention differs from what currently exists. Although there are other devices that can connect a tubular attachment to a Vaporizer 1, and they can do so while forming a substantially airtight connection, none of these currently available devices provide for a means to operate the power button 6 on the Vaporizer 1. In fact, other devices make it more difficult to operate the power button 6 because they block access to the power button 6 once they are installed. Therefore, unlike the current invention, devices that currently exist must be removed from the Vaporizer 1 every time the user wishes to depress the power button 6. This is especially important when the Vaporizer's 1 functions or modes can be controlled through alternative actions applied to the power button 6, such as depressing the power button 6 multiple times in a row, or depressing and holding down the power button 6 for a predetermined period of time. Under such circumstances, having continued access to the power button 6 is essential to the proper and complete operation of the Vaporizer 1 and is therefore a very important feature of the invention 10.

FIGS. 1 through 7 have been provided to illustrate a potential embodiment of the invention 10, as well as discloser its interaction with the type of Vaporizer 1 it is meant to modify. FIG. 1 shows the Vaporizer 1 with an installed standard mouthpiece 2 from an upper corner, perspective view. The mouthpiece 2 has two openings 3 through which the user inhale vapor. The mouthpiece has a Pressure Point 4. The Pressure Point 4 is a membrane 4 covering a peg 5 that is driven down into the power button 6 when a user depresses the Pressure Point 4.

FIG. 2 shows a cross-section side view of the Vaporizer 1 with a detailed depiction of internal structures near the top where it interfaces with the mouthpiece 2, and near the bottom where the heating chamber 8 holds the material to be vaporized. Otherwise, FIG. 2 omits internal structures that are irrelevant to the interaction of the Vaporizer 1 with the invention 10. A void 31 at the bottom of the mouthpiece 2 connects the two openings 3 through which the user inhales vapor to the airpath 7 of the Vaporizer 1.

The Pressure Point 4 allows a user to depress the peg 5, activating the power button 6.

FIG. 3 is a perspective view of the Vaporizer 1 with a standard mouthpiece 2 separated from the base 36 of the Vaporizer 1. The mouthpiece 2 has an upper surface 39 and an outer side surface 38. The outer side surface 38 of the mouthpiece 2 snuggly mates with a continuous inner side surface 33 of the base 36. The inner top surface 37 of the base 36 has an airpath 7 and a silicon membrane 9 covering the power button 6. The mouthpiece 2 sits flush with the rim 35 of the base 36.

The mouthpiece 2 sits flush with the rim 35 of the base 36.

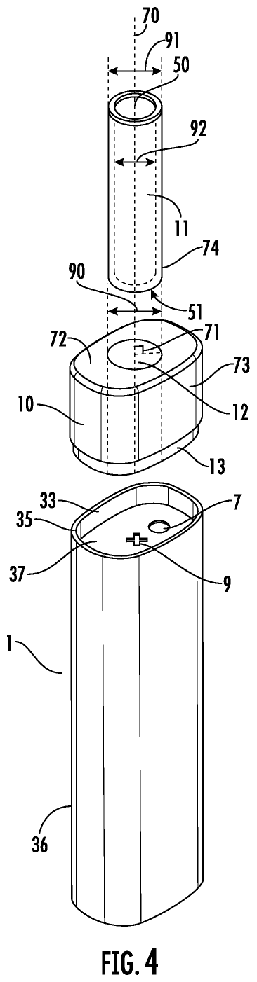

FIG. 4 is a perspective view of the Vaporizer 1 base 36 with the invented Adapter 10 and an Attachment 11. The Vaporizer 1 base 36 has the inner side surface 33; the inner top surface 37; and rim 35. The airpath opening 7 and silicon membrane 9 covering the power button 6 are located on the inner top surface 37.

The Attachment 11 is a hollow cylinder having an outer diameter 91, an inner diameter 92, an opening at the top 50, and an opening at the bottom 51.

In the embodiment shown here, the Adapter 10 has a cylindrical void (“Attachment Interface”) 12 in its top surface 72. The Attachment Interface has an inner diameter 90 at the point it meets the top surface 72. The Attachment Interface 12 is coated with an elastomer. The inner diameter 90 of the Attachment Interface 12 is nominally equal to the outer diameter 91 of the Attachment 11. This insures an air-tight fit between the Attachment 11 and the Adapter 10. The Attachment Interface 12 has a longitudinal axis 70 that it shares with at least the end of the mating end 74 of the Attachment 11. The longitudinal axis 70 forms an angle 71 with the top surface 72 of the Adapter 10. In this embodiment, the angle is 90°, but the angle 71 can practically range from about 45° to 90°. The Adapter 10 has an upper side surface 73. The Adapter 10 has a lower side surface 13 that fits inside the inner side surface 33 of the Vaporizer 1 base 36.

FIG. 5 is a lower perspective view, showing the bottom surface 40 of the Vaporizer 1 base 36 and the bottom surface 80 of the Adapter 10. The Attachment Interface 12 comes out the bottom surface 80, making the Attachment Interface 12 a through-hole. In this embodiment, the Attachment Interface 12 is cylindrical. The Attachment Interface 12 can also be a frustoconical with a very slight taper from the top surface 72 to the bottom surface 80. The inner surface of the Attachment Interface 12 is coated with an elastomer. The lower side surface 13 of the Adapter has a silicon O-ring 60 to create the air-tight fit between the Adapter 10 and the base 36. In an alternative embodiment, the lower side surface 13 is tapered and coated with an elastomer, which also creates an air-tight fit between the Adapter 10 and the base 36 of the Vaporizer 1. In this embodiment, the minimum perimeter of the lower side surface is immediately adjacent to the bottom surface.

The Adapter 10 houses a rigid insert 15 that has an actuator 18. The rigid insert 15 is internally adjacent to the bottom surface 80 with the actuator 18 protruding. The bottom surface 80 of the Adapter 10 has a void 14 to create a continuous air-flow channel between the airpath 7 and the Attachment 11.

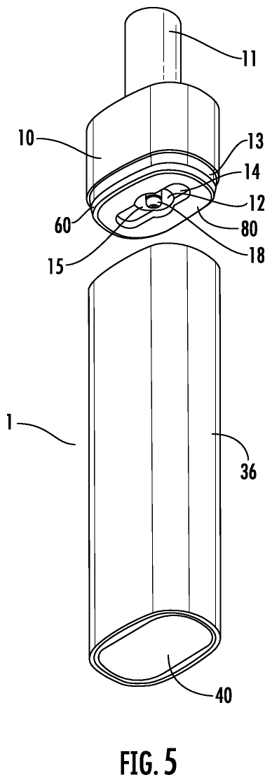

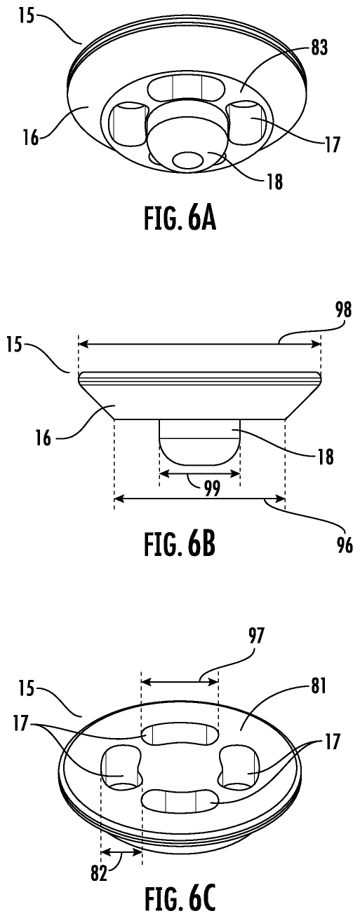

FIGS. 6A-6C show the lower perspective, side, and upper perspective views of the rigid insert 15. The rigid insert 15 has an upper surface 81 with a plurality of through-holes 17. The through-holes 17 have an overall length 97 and an overall width 82. In the embodiment shown in FIGS. 6A-6C, the through-holes 17 are elongated and kidney shaped, although the through-holes 17 can be made from any shape. The rigid insert 15 has an outer diameter 98. The rigid insert 15 has a beveled side surface 16, a lower surface 83, and an actuator 18. The lower surface 83 has an outer diameter 96. Likewise, the actuator 18, has an outer diameter 99.

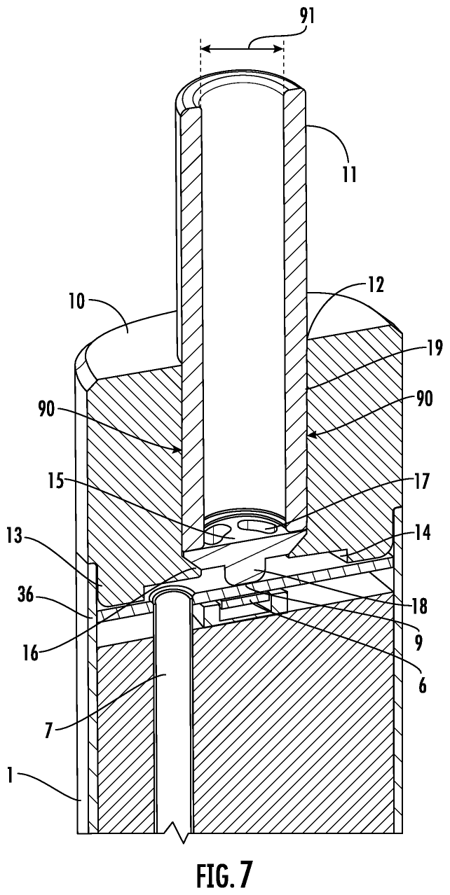

FIG. 7 is a cross section showing the interaction of the Attachment 11, the adapter 10, and the Vaporizer 1. A constant air-flow channel is created by the inner diameter 91 of the Attachment 11 which connects to the plurality of through-holes 17 of the rigid insert 15, allowing air to flow into the void 14 and then into the airpath 7. The Attachment 11 has an outer diameter 90 allowing it to fit snuggly into the Attachment Interface 12 of the Adapter 10. The Attachment Interface 12 is a cylindrical void 12 that is coated with an elastomer 19 in order to create an air-tight seal. The actuator 18 is positionally adjacent to the silicon membrane 9 covering the power button 6. A user can press down on the Attachment 11 into the rigid insert 15, causing the actuator 18 to depress the power button 6 through the silicon membrane 9. The lower side surface 13 of the Adapter 10 fits snuggly into the base 36 of the Vaporizer 1.

The figures show a progression of how the invention 10 works with the Attachment 11 and the Vaporizer 1. FIG. 5 shows the Attachment 11 attached to the Adapter 10, but not yet attached to the Vaporizer 1. FIG. 7 shows the Attachment 11 and an Adapter 10 fully installed on the Vaporizer 1.

Additional description of the various elements discussed here is as follows:

Attachment Interface. The Attachment Interface 12 is a cylindrical void that has a inner diameter 90 nominally equal to the outer diameter 91 of the Attachment 11.

In this embodiment, the Attachment Interface 12 vertical with an opening facing upward, and the entirety of the internal surface is made up of an elastomeric material. Ribs (not shown) can optionally be molded into the elastomeric material. The bottom of the Attachment Interface supports the rigid insert 15.

Vaporizer Interface. The Vaporizer Interface is a structure that complements and attaches the Adapter 10 to the Vaporizer 1. The lower side surface 13 of the Adapter 10 has a silicon O-ring 60 to create the air-tight fit between the Adapter 10 and the base 36 of the Vaporizer 1. The lower side surface 13 is made of an elastomeric material. The lower side surface 13 is designed to have a nominal interference fit with the inner side surface 33 of the base 36 of the Vaporizer 1. In an alternative embodiment, the lower side surface 13 of the Adapter 10 is tapered, starting slightly larger than the inner side surface 33 of the Vaporizer 1 and terminating so that it is slightly smaller than the inner side surface 33 of the Vaporizer 1 base 36.

Rigid Insert. The rigid insert 15 is a rigid, button-like structure that has an upward facing surface 81 that has an outer diameter 98 that is larger than the outer diameter 91 of the Attachment 11. The rigid insert 15 has a lower surface 83 with an outer diameter 96 that is at least as large as the inner diameter 92 of the Attachment 11.

In alternative embodiments of the invention, some of the above features may be absent or may be present in different forms. Below is a discussion of some, but not all, of these possible variations.

The Attachment Interface 12 may vary. The direction of the Attachment Interface 12 does not have to be perpendicular to and may be at an angle to the top of the Adapter 10. In one alternative embodiment, the longitudinal axis of the Attachment Interface 12 may be at a 45-degree angle to the upper surface of the Vaporizer 1.

Additional embodiments can be made by modifying the material that forms the sides of the Attachment Interface 12. The material that forms the sides of the Attachment Interface 12 may be a mixture of elastomeric and rigid materials. For example, a portion of the cylinder constituting the Attachment Interface 12 may have larger in diameter than that of the Attachment 11. Elastomeric ribs may then be embedded in the Attachment Interface 12 in order to create an interference fit between the ribs and the Attachment 11. Conversely, the entirety of the portion of the cylinder constituting the Attachment Interface 12 may be made of elastomeric material that causes a nominal interference fit with the Attachment 11.

Regardless of the embodiment, there are two important relationships between the components. The first relationship creates a continuous route for the air/vapor to travel through as it moves from the Vaporizer 1 to the user's mouth. This occurs when the Vaporizer Interface creates a non-permanent, but substantially air-tight connection between the Adapter 10 and the Vaporizer 1 and directs the air through the airpath 7, void 14, through-holes 17 in the rigid insert 15, and through the Attachment 11.

The second relationship creates a continuous kinetic link between the Attachment 11 and the power button 6. This is accomplished because the Attachment Interface 12 holds the Attachment 11 in contact, or nearly in contact, with the rigid insert 15 which itself is designed to transfer kinetic energy from the Attachment 11 down through its Actuator 18 through the Silicone Membrane 9 and into the power button 6.

A user uses the Adapter 10 by pressing an Attachment 11 into the Attachment Interface 12 and then mounting the Adapter 10 to the Vaporizer 1 Base 36. The user can cycle power by pressing down on the Attachment 11 which will cause the actuator 18 to depress the power button 6. The user can use the power button 6 to control other functions if the power button 6 is so enabled. The user can then draw vapor from the heating chamber 8 through the airpath 7, the void 14, the through-holes 17 in the rigid insert 15, the Attachment Interface 12 and the Attachment 11.

We claim:

1. An adapter for vaporization attachments comprising
a top surface;
an upper side surface;
a lower side surface;
a bottom surface;
an attachment interface; and
a rigid insert having an upper surface, a lower surface, a plurality of through-holes, a beveled side surface, and an actuator;
wherein the attachment interface is a through-hole from the top surface to the bottom surface (Attachment Interface);
wherein the inner surface of the Attachment Interface is coated with an elastomer;
wherein the Attachment Interface has an inner diameter at the top surface;
wherein the inner diameter of the Attachment Interface at the top surface is nominally equal to an outer diameter of the mating end of an attachment; and
wherein the mating end of an attachment can be inserted into the Attachment Interface, creating an air-tight seal.

2. The adapter for vaporization attachments in claim 1, wherein the adapter can be mounted to a base of a vaporizer.

3. The adapter for vaporization attachments in claim 2, wherein the lower side surface of the adapter is coated with an elastomer.

4. The adapter for vaporization attachments in claim 3, wherein the lower side surface of the adapter fits within a continuous inner side surface of the base of the vaporizer, making an air-tight seal.

5. The adapter for vaporization attachments in claim 4, wherein the lower side surface is tapered with its minimum perimeter immediately adjacent to the bottom surface.

6. The adapter for vaporization attachments in claim 4, wherein the lower side surface has an O-ring.

7. The adapter for vaporization attachments in claim 4, wherein the bottom surface of the adapter has a void.

8. The adapter for vaporization attachments in claim 7, wherein the rigid insert is positioned internally adjacent to the bottom surface of the adapter, so that the actuator protrudes and the through-holes are adjacent to the void in the bottom surface.

9. The adapter for vaporization attachments in claim 8, wherein the vaporizer has a heating chamber and an airpath leading from the heating chamber; and wherein the airpath leading from the heating chamber, the void in the bottom surface of the adapter, the through-holes, the Attachment Interface, and the attachment create a single air-flow channel through which a user is able to draw vapor.

10. The adapter for vaporization attachments in claim 9, wherein the upper surface of the rigid insert has an outer diameter which is equal to or greater than the outer diameter of the mating end of the attachment.

11. The adapter for vaporization attachments in claim 10, wherein the Attachment Interface has a longitudinal axis that it shares with at least the mating end of the attachment.

12. The adapter for vaporization attachments in claim 11, wherein the longitudinal axis makes an angle with the top surface of the adapter.

13. The adapter for vaporization attachments in claim 12, wherein the angle so made is 90°.

14. The adapter for vaporization attachments in claim 12, wherein the angle so made is between 45° and 90°.

15. The adapter for vaporization attachments in claim 12, wherein the Attachment Interface is cylindrical.

16. The adapter for vaporization attachments in claim 12, wherein the Attachment Interface is a frustoconical with a slight taper from the top surface of the Adapter to the bottom surface of the Adapter.

17. The adapter for vaporization attachments in claim 12, wherein the vaporizer has a power button and wherein pressing the attachment towards the vaporizer base causes the actuator to depress the power button of the vaporizer.

18. The adapter for vaporization attachments in claim 12, wherein the through-holes in the rigid insert are elongated and kidney-shaped.
